(19) United States
(12) Patent Application Publication
(10) Pub. No.: US 2015/0322467 A1
(43) Pub. Date: Nov. 12, 2015

(54) METHOD FOR OPTIMIZING PRODUCTION OF EICOSAPENTAENOIC ACID (EPA) IN A RECOMBINANT HOST

(71) Applicant: SCFM VENTURES, LLC, TULSA, OK (US)

(72) Inventor: ANDREE F. ELLIOTT, Longview, TX (US)

(21) Appl. No.: 14/708,177

(22) Filed: May 8, 2015

Related U.S. Application Data
(60) Provisional application No. 61/990,566, filed on May 8, 2014.

(51) Int. Cl. C12P 7/64

(52) U.S. Cl. CPC C12P 7/6427

(57) ABSTRACT

The present invention relates to a method for optimizing production of eicosapentaenoic acid (EPA) production by cloning genes into a bacterial host, most preferably a modified Escherichia coli strain. Four polyunsaturated fatty acid (PUFA) producing genes native to the cold water Pacific bacterium Shewanella pneumatophori SCRC-2738 and one from Moritella marina are cloned into an E. coli strain modified for increased EPA output. The heterologous enzymes function according to the Polyketide Synthesis (PKS) pathway not known to occur natively in E. coli. Certain modifications to the E. coli strain to increase yield include: culturing considerations; inactivating the native E. coli genes that control fatty acid biosynthesis, fatty acid degradation, and acetyl-CoA consumption; and inserting genes to augment cellular production of NADPH, acetyl-CoA, malonyl-CoA and phosphopantetheinyl transferase and inserting chaperonin genes to allow the E. coli to grow at a normal rate at lower temperatures.

### CROSS REFERENCE TO RELATED APPLICATION

This application claims the benefit of U.S. Provisional Application No. 61/990,56 filed May 8, 2014, herein incorporated by reference in its entirety for all purposes.

### FIELD OF THE INVENTION

The present invention relates, generally, to the production of Eicosapentaenoic Acid (EPA). More specifically, the present invention relates to the optimization of EPA production by cloning genes in bacterial host cells.

### BACKGROUND OF THE INVENTION

A few decades ago, eukaryotes alone were thought to produce polyunsaturated fatty acids, or PUFAs. However, it was discovered that certain prokaryotes, especially psychrophiles and/or piezophiles also produced these lipids and other researchers began isolating and culturing these strains. These cold water marine cells incorporate the PUFAs into phospholipids in the cellular membrane to lower the lipid freezing point, giving the membrane added fluidity and flexibility at cold temperatures. Meanwhile in the food and health industries, more research illuminated the health benefits of PUFAs, and specifically omega-3 fatty acids. In recent years, taking omega-3 fatty acids such as EPA as a dietary supplement or preventative/therapeutic agent has been gaining momentum and is expected to continue.

Industrial production of EPA. In industry to date, oil is extracted from fish using an organic solvent system, after which the PUFA fraction of the fish oil is concentrated or enriched. Further purification can be achieved through chromatographic methods such as thin layer chromatography, which separates the fatty acids based on the degree of unsaturation. For the pharmaceutical industry, the fatty acid ethyl ester (or FAEE) is the preferred and most common form.

Other attempts have been made to produce EPA from genes cloned into bacterial cells. However, such attempts have fallen short in producing such EPA on a commercially viable scale.

A need, therefore, exists for a method of production of EPA which does not require extraction from fish. A need further exists for a method of production which produces EPA from cloned genes into bacterial cells in a pure form which can be optimized for commercially viable pharmaceutical, commercial and industrial applications.

### SUMMARY OF THE INVENTION

The present invention relates to a method for optimizing of eicosapentaenoic acid (EPA) production by cloning genes into a bacterial host. For the purpose of the present disclosure the term “recombinant bacterial host” shall mean an organism containing DNA from different microorganisms, most of which are genes which will be expressed. A preferred recombinant bacterial host is Escherichia coli (E. coli) as herein described. A particularly suitable strain of E. coli is NEB-1013, available from New England Biolabs, Inc., Ipswich, Mass.

Four polyunsaturated fatty acid (PUFA) producing genes native to the cold water Pacific bacterium Shewanella pneumatophori SCRC-2738 and one from Moritella marina MP-1 are cloned into an E. coli strain modified for increased EPA output. A goal being to produce EPA on a large (commercial) scale. The heterologously produced enzymes function according to the Polyketide Synthesis (PKS) pathway not known to occur natively in E. coli. Certain modifications to the E. coli strain to increase yield include, but are not limited to: culturing considerations; inactivating the native E. coli genes that control fatty acid biosynthesis, fatty acid degradation and acetyl-CoA consumption; and inserting genes to augment cellular production of NADPH, acetyl-CoA, malonyl-CoA and phosphopantetheinyl transferase, and inserting chaperonin genes to allow the E. coli to grow at a normal rate at lower temperatures (i.e. 15° C. as opposed to 37° C.). The E. coli genomic changes will be made to improve the yield of EPA produced by the heterologously expressed genes.

A method of producing eicosapentaenoic acid (EPA) in a recombinant bacterial host according to the present disclosure may include in one embodiment: a.) selecting a bacterial host including at least one biosynthetic pathway, at least one degradation pathway, and at least one metabolic pathway; then, i.) deleting the at least one biosynthetic pathway; ii.) deleting the at least one degradation pathway; and, iii.) deleting the at least one metabolic pathway; b.) inserting genes to the bacterial host selected from a group consisting of Escherichia coli panK, Bacillus subtilis sfp, Moritella marina putative thioesterase I, Moritella marina pfaE, Pseudoalteromonas sp. GroEL, and E. coli GroES to produce a recombinant host; c.) expressing in said bacterial host genes cloned into a first pBAD; said genes selected from the group consisting of Shewanella pneumataphori SCRT-2738 pfaA, pfaB, pfaC, and pfaD; d.) expressing in said bacterial host Moritella marina MP-1 pfaE gene cloned into a second pBAD; and, e.) growing the recombinant host to optimize EPA production.

In a preferred embodiment the bacterial host is Escherichia coli (E. coli). The recombinant host may be grown at low temperatures and may be cultured in corn steep liquor. For the purpose of the present disclosure, the term low temperature(s) shall mean temperatures below approximately 16° C., preferably between 13° C. and 16° C.; and more preferably between 14° C. and 15° C. and most preferably at approximately 14° C. and/or 15° C.

In a particularly preferred embodiment the biosynthesis pathway is a fatty acid biosynthesis gene such as fabB; the degradation pathway is a fatty acid degradation gene, and most preferably fadD and fadE; and the metabolic pathway is a phosphate acetyl transferase gene most preferably the E. coli pta gene and/or the E. coli pgi gene. The knockout of the E. coli pta gene may be accompanied with the over-expression of the panK gene.

### BRIEF DESCRIPTION OF THE DRAWINGS

FIG. 1 is a model of the λ-red plasmid constructed according to the method of the present disclosure.

FIG. 2 is a model of the donor plasmid constructed according to the method of the present disclosure.

FIG. 3 is a model of the helper plasmid constructed according to the method of the present disclosure.

### DESCRIPTION OF THE PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processes and manufacturing techniques are omitted so as to not unnecessarily obscure the embodiments herein. The examples used herein are intended merely to facilitate an understanding of ways in which the invention herein may be practiced and to further enable those of skill in the art to practice the embodiments herein. Accordingly, the examples should not be construed as limiting the scope of the claimed invention.

Modifications in the bacterial host. The following describe deletions and insertions/over-expressions in the bacterial host. Laboratory strains of Escherichia coli are known for being rapid and nonfastidious growers, making it an excellent host for heterologous genes and gene products. However, without amending the host metabolism to augment recombinant gene expression, only a minimal amount of EPA is produced. The following proposed modifications, both deletions and insertions, will be pursued to increase EPA yield in recombinant E. coli cells. Certain modifications to the E. coli strain to increase yield include, but are not limited to: culturing considerations; inactivating the native E. coli genes that control fatty acid biosynthesis, fatty acid degradation and acetyl-CoA consumption; and inserting genes to augment cellular production of NADPH, acetyl-CoA, malonyl-CoA and phosphopantetheinyl transferase, and inserting chaperonin genes to allow the E. coli to grow at a normal rate at lower temperatures.

Gene deletions/knockout. First described are the E. coli genes selected for gene deletion/knockout in order to increase EPA production.

Delete/knockout fatty acid biosynthesis pathway. Host Escherichia coli cells with the fatty acid biosynthesis fabB gene knocked out are unable to produce their own unsaturated fatty acids (PUFAs) and thus accumulate higher concentrations of recombinant EPA. It is known in the art to increase EPA yield with the addition of the antibiotic cerulenin to a culture of recombinant E. coli. Cerulenin works by inhibiting the β-ketoacyl-acyl protein synthetase, the enzyme that catalyzes the condensation reaction of fatty acid biosynthesis, specifically the fabB gene. This confirms that the fabB gene should be knocked out.

Delete/knockout fatty acid degradation pathways. Knocking out the fadD and fadE genes will also increase PUFA production by altering the initiation of the fatty acid degradation pathway. The fadD gene codes for the fatty acyl-CoA synthase, the first step of the 13-oxidation pathway for degradation. The fadE gene codes for acyl-CoA dehydrogenase, the enzyme that catalyzes the beta-oxidation of the acyl-CoA that was synthesized by the fadD gene product. By knocking out these genes, the degradation pathway is turned off, which results in improved yields of total fatty acids.

Delete/knockout metabolic pathways. Knocking out E. coli pgi and E. coli pta genes will increase PUFA production by altering the initiation of metabolic pathways. Knocking out the E. coli gene pgi (which codes for glucose-6-phosphate isomerase) has been shown to increase NADPH concentration and optimize product (e.g., EPA) yield. Coupled with over-expression of the E. coli panK gene, deletion of E. coli pta increases coenzyme-A (CoA) and acetyl-CoA production. The pta gene catalyzes the first step of the pathway to convert acetyl-CoA to acetate, thus the increase in acetyl-CoA, which is a substrate in the production of EPA.

Increase production of NADPH. EPA synthesis is specifically dependent on NADPH/NADP⁺ and does not require NADH. NADPH is a known reducing agent. Supplementation of recombinant E. coli cells with NADPH in the media will increase yields of heterologously produced polyunsaturated fatty acids (PUFAs), such as EPA. Because supplementation of media with NADPH would be cost prohibitive, host E. coli cells could instead be engineered by gene deletion to increase the intracellular levels of NADPH. Knocking out the E. coli gene pgi (which codes for glucose-6-phosphate isomerase) has been shown to increase NADPH concentration and optimize product (e.g., EPA) yield. Knocking out pgi directs the entire uptake of glucose into the pentose phosphate pathway (PPP) rather than through the glycolytic pathway. Glucose-6-phosphate (G-6-P) is the product of the first step of glycolysis; redirection of G-6-P to the PPP results in a high NADPH generation rate. It has also been observed that NADPH overabundance in Δpgi cells drives excessive fatty acid biosynthesis translating the redox imbalance into an imbalance in fatty acid production. This is a positive outcome, since EPA is a fatty acid. Note that when pgi is knocked out, flux through the glyoxylate shunt is increased and acetate secretion is decreased. The glyoxylate shunt is a variation of the tricarboxylic acid (TCA) cycle in E. coli and centers on the conversion of acetyl-CoA to succinate for the synthesis of carbohydrates. It is because of the glyoxylate shunt that Δpgi cells remain viable.

Knocking out the phosphate acetyl transferase (E. coli pta) gene will also increase PUFA production by altering a metabolic pathway. The deletion of the E. coli pta gene coupled with the over-expression of the panK gene increase CoA production by 313% and acetyl-CoA production by 227%. The pta gene catalyzes the first step of the pathway to convert acetyl-CoA to acetate, thus decreasing the acetyl-CoA pool when the pta gene product is active. When the pta gene is inactivated, the acetyl-CoA pool is increased but the acetate pathway deletion mutants are weaker and grow slower in anaerobic conditions; thus, these cells should be grown aerobically.

Strategies for gene knockout in a preferred embodiment. The preferred method for gene knockout is comprised of two parts: (1) Use of a web-based design tool to generate translational gene knockout sequences; and (2) insertion of genes to be over-expressed into the gene to be knocked out. Each method is described below.

A web-based tool, MODEST (MAGE Oligo Design Tool, where MAGE=multiplex automated genome engineering), is a web-based tool available to automatically design oligos based on desired genotypic or phenotypic changes and is available for free and is open to all users at http://modest.biosustain.dtu.dk. Bonde, M. T., Klausen, M. S., Anderson, M. V, Wallin, A. I. N., Wang, H. H., & Sommer, M. O. a. (2014). MODEST: a web-based design tool for oligonucleotide-mediated genome engineering and recombineering. Nucleic Acids Research, 42 (Web Server issue), W408-15. doi:10.1093/nar/gku428, Online version. This online tool can be used to introduce tandem stop codons in the first half of a gene sequence. By introducing three tandem stop codons, the probability of restoring gene function by reverse mutations is reduced because all of the stop codons would need to be simultaneously reverted. The tandem stop codons are designed to use all three types of stop codons to abolish the possibility of restoring gene function via a tRNA suppressor mutation. By localizing the mutations to the first half of the gene, the likelihood of producing a truncated protein with some residual activity is minimized. The MODEST web-based tool will be used to design the pgi, fabB, and pta knockout gene sequences. The E. coli genomic sequence of each of these genes will undergo recombination, using the recombineering method, to replace the wild-type pgi, fabB, and pta sequences with the MODEST-altered translational gene knockout sequence.

The fadD gene will be knocked out using the recombineering method to insert the Pseudoalteromonas sp. PS1M3 groEL chaperonin gene and the E. coli groES chaperonin gene sequences into the E. coli genome at the fadD loci. These two gene sequences will be preceded by E. coli promoter sequences for constitutive over-expression of these chaperonin genes.

The fadE gene will be knocked out using the recombineering method to insert a gene cluster consisting of the M. marina putative thioesterase gene, the panK gene, and the B. subtilis sfp gene into the E. coli genome at the fadE loci. These gene sequences will be preceded by promoter sequences for constitutive over-expression of the genes in this cluster.

Gene insertions/additions to the bacterial host. Insertions/additions to the bacterial host pursuant to the present disclosure shall next be described.

At the same time as making deletions in the host genome to prevent degradation or metabolic depletion of desired products and substrates, certain genes will also be added to the host genome. Some or all of these heterologous genes will be synthesized to increase their desired activity. These genes will be assembled into one or more clusters with an arabinose promoter using the Gibson Assembly method, modified Gibson Assembly method, or other similar means and inserted into the host genome using recombineering techniques. These genes include E. coli panK, Bacillus subtilis sfp, Moritella marina putative thioesterase I, Pseudoalteromonas sp. GroEL, and E. coli GroES described in greater detail below. The M. marina putative thioestersase I gene will be cloned in the same cluster with Bacillus subtilis sfp and E. coli panK.

To express the PUFA producing genes, Shewanella pneumataphori pfaA, pfaB, pfaC, and pfaD and Moritella marina pfaE are cloned into two respective pBAD plasmids. In a preferred embodiment, the first pBAD plasmid includes Shewanella pneumataphori SCRT-2738 pfaA, pfaB, pfaC, and pfaD genes. The second pBAD plasmid includes the Moritella marina MP-1 pfaE gene.

An arabinose promoter will allow for the over-expression of the genes in the clusters. This applies to the E. coli panK, and Bacillus subtilis sfp and the Moritella marina putative thioesterase I, genes. E. coli σ⁷⁰ and σ³² promoters will allow for the constitutive expression of the chaperonin genes in the cluster. This applies to the Pseudoalteromonas sp. GroEL, and E. coli GroES chaperonin genes of the preferred embodiment.

Insertion/over-expression of Escherichia coli panK in the bacterial host. The E. coli panK gene codes for the pantothenate kinase protein. This enzyme is responsible for phosphorylating pantothenic acid (vitamin B5) to produce coenzyme-A (CoA). Cellular production of EPA begins with an acetyl-CoA substrate. Both the fatty acid synthesis and the polyketide synthesis pathways utilize acetyl-CoA and malonyl-CoA as substrates, which are both derived from the cofactor coenzyme-A (or CoA). Pantothenic acid (vitamin B5, formerly known as vitamin B3) is phosphorylated to produce CoA. This phosphorylation, which is the rate-controlling step in CoA production, is catalyzed by the enzyme pantothenate kinase, encoded by the E. coli gene panK. Over-expression of this gene with concomitant media supplementation of pantothenic acid (5 mM) results in a significant increase of CoA and acetyl-CoA levels in E. coli cells. Acetyl-CoA and malonyl-CoA are the substrates from which EPA is produced; both are derived from the phosphorylation of coenzyme-A. To over-express the pantothenate kinase enzyme, the panK gene would be incorporated into to gene cluster and inserted into the host genome. The concomitant pantothenic acid supplementation could be accomplished by growing the cells in corn steep liquor, which has been shown to have a high B5 vitamin content. The growing media will be discussed in greater detail below.

As will be apparent to one of skill in the art, the over-expression of the E. coli panK gene and the expression of Moritella marina pfaE (described below) provide the same result. However, it was determined that over-expression of the E. coli panK alone did not produce suitable amounts of EPA. In a preferred embodiment, both are accomplished in order to optimize EPA production according to the present disclosure.

Insertion/expression of Bacillus subtilis sfp in the bacterial host. The sfp gene from Bacillus subtilis codes for the phosphopantetheinyl transferase (PPTase) protein. The sfp gene from Bacillus subtilis is thought to be the most tolerant in effectively modifying ACP's from all PKS (polyketide synthesis) subclasses. Expression of the sfp PPTase gene in either a plasmid-borne or chromosomal format has been shown to result in stoichiometric pantetheinylation of soluble PKS proteins in E. coli. The PPTase enzyme catalyzes the transfer of a phosphopantetheinyl group from intracellular CoASH (reduced coenzyme A) to the active site of an acyl carrier protein (ACP); this is the first step in the cellular production of fatty acids, including EPA. The extent of this post-translational modification of ACP within cells controls the rate and yield of produced fatty acids. Cloning this heterologous gene (sfp) into E. coli cells will produce more PPTase enzyme with the goal of increasing EPA production

Insertion/overexpression of Moritella Marina putative thioesterase I in the bacterial host. The Moritella marina gene codes for a thioesterase I protein. Thioesterase activity is typically required for the release of products from polyketide synthase enzymes in the production of polyunsaturated fatty acids (PUFAs). Typically, the gene for thioesterase in deep-sea bacteria, including Shewanella pneumatophori SCRC-2738, has not been found within the PUFA synthase gene cluster (pfaA-D). However, a gene (orf6) that codes for a putative “hotdog” thioesterase has been identified in the PUFA synthase gene cluster of the psychrophilic marine organism Photobacterium profundum SS9. Thioesterases are classified into two distinct protein families: the α/β-hydrolase thioesterase and the hotdog thioesterase. The hotdog thioesterase was first described as a seven-stranded antiparallel β-sheet that resembles a “bun” that wraps around the five-turn α-helical “sausage”. Typically, the hotdog thioesterases are associated with the hydrolysis of aromatic thioesters, but the P. profundum Orf6 thioesterase shows a preference for eicosapentaenoyl-CoA, which is the thioester present in the production of EPA. In a BLAST database search for Orf6 homologs, a similar ortholog was found in M. marina (86% identical, 0 gaps). And, like the P. profundum orf6 gene which is located directly upstream of the pfaA gene of its PUFA synthase complex, the orf6 ortholog in M. marina is located within 1000 bases of the start of its pfaA gene. It has therefore been suggested that these thioesterases are involved in PUFA release. Because it has also been shown that, in marine bacteria, fatty acid release by a thioesterase is a limiting factor in the production of fatty acids, it is proposed that the over-expression of the orf6 ortholog of M. marina be cloned into the genome of the E. coli host cells to increase the yield of EPA produced in E. coli.

Insertion/expression of GroEL and GroES genes in the bacterial host. The Pseudoalteromonas sp. PS1M3 GroEL and E. coli NEB 10-β GroES chaperonin genes will also be expressed in the E. coli host cells in the preferred embodiment. The purpose of expressing these proteins in the host cells is to enable the cells to grow well at 15° C. (low temperatures). Presence of the cold-adapted (GroEL) chaperonin should shorten the E. coli doubling time and minimize inclusion body formation of the heterologously-produced proteins when the cells are grown at 15° C. The result should be the production of EPA, since the EPA-producing enzymes will be present in the active form, as opposed to the inactive, aggregated inclusion body form.

Strategies for gene insertion/expression/overexpression in a preferred embodiment. The above described sets of individual gene sequences will be ligated together into one contiguous sequence, referred to herein as the gene cluster. This can be repeated for multiple gene clusters. Two gene clusters will be produced to be incorporated into the E. coli genome: one cluster for the over-expression of panK, M. marina putative thioesterase gene, and sfp and one cluster comprised of the two chaperonin genes. Essentially, the method to be used for ligation and formation of the gene clusters was established by Daniel G. Gibson and is thus referred to as the Gibson Assembly method. The specific protocol used in ligating individual gene sequences into gene clusters in this project is the NEBuilder® HiFi DNA Assembly kit (New England Biolabs). The process hereafter, however, will continue being referred to as the Gibson Assembly method.

Recombineering for the insertion of gene clusters into the E. coli genome. The gene clusters produced by Gibson Assembly will be incorporated into the E. coli genome for the purpose of gene expression and, in the preferred embodiment, for the purpose of knocking out genes whose products limit EPA production (as described above). The method to be used for incorporating (inserting) gene clusters into genomic DNA, is a form of “recombineering,” This method involves the construction of three plasmids which are transformed into competent E. coli host cells: A λ-red plasmid, a donor plasmid, and a helper plasmid. The composition and function of each plasmid is described below.

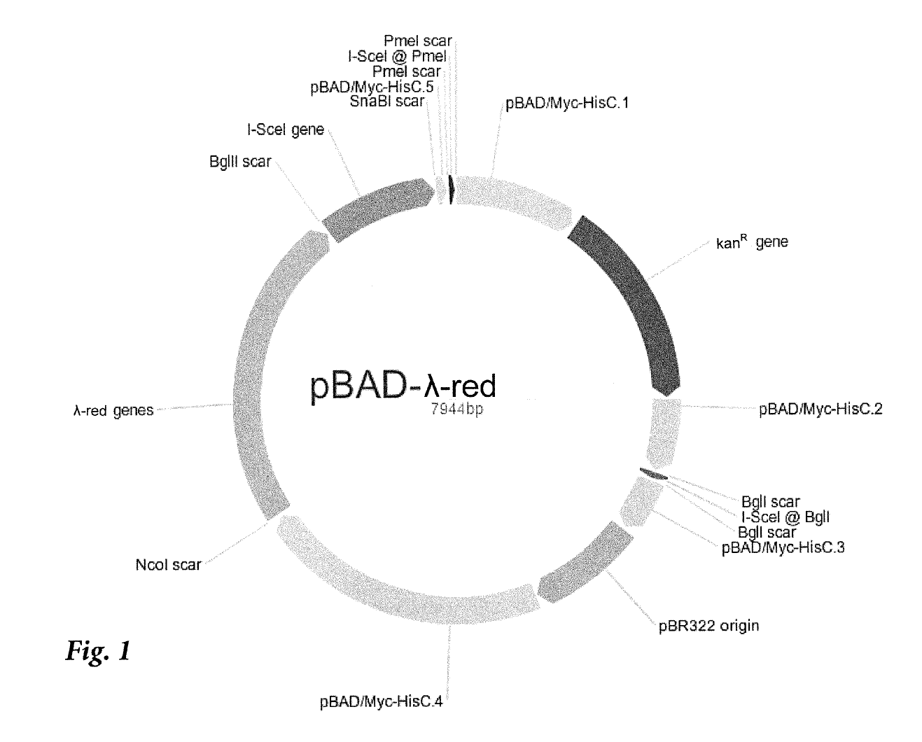

The λ-red plasmid. This plasmid, constructed from pBAD, includes the phage λ recombination genes, in a preferred embodiment, the gam gene, the bet gene, and the exo gene. These genes will next be described. A model for how recombineering occurs for the λ-red plasmid is set forth in FIG. 1.

The gam gene codes for the Gam protein. This protein prevents an E. coli nuclease, RecBCD, from degrading linear DNA fragments, allowing preservation of transformed linear DNA in vivo.

The bet gene codes for the Bet (or Beta) protein. Bet is a ssDNA-binding protein that promotes annealing of two complementary DNA molecules.

The exo gene codes for the Exo protein. This protein has 5′-to-3′ dsDNA exonuclease activity.

Working together, these latter two λ-red proteins insert linear DNA (the gene cluster) into the desired target (E. coli genomic DNA in the preferred embodiment) creating a genetic recombinant. For dsDNA, λ-red Exo is thought to degrade linear DNA (the gene cluster) from both 5′-ends, exposing ssDNA that is bound by λ-red Beta. Models for how recombineering occurs propose that the single-stranded regions of the incoming linear DNA (gene cluster) bound by the Beta protein are annealed to complementary single-stranded gaps arising at the replication fork during DNA replication. Consistent with this model, an oligo that is able to anneal to the discontinuously replicated lagging strand gives a higher recombination frequency than its complementary leading strand oligo. See FIG. 1.

Also included in the λ-red plasmid is the Saccharomyces cerevisiae I-SceI gene, which codes for the I-SceI protein. This protein is an endonuclease with very high sequence specificity; it recognizes a non-symmetrical, 18-bp sequence (the I-SceI recognition site) and generates a four base pair staggered cut with 3′-OH overhangs. The catalytic activity of I-SceI is used to excise the gene cluster/knockout gene sequence from the donor plasmid, which includes two I-SceI recognition sites. This sequence excision from the donor plasmid makes the dsDNA gene cluster/knockout gene sequence available for insertion into the E. coli genome. It is the action of the λ-red recombination proteins that are then responsible for the sequence insertion into the E. coli genome. There are also two I-SceI recognition sites located in the λ-red plasmid, which allow for self-cleavage of this plasmid. The purpose of cleaving the λ-red plasmid is to prevent over-expression of the 80 -red proteins and to eliminate unnecessary plasmids from the host cells.

The λ-red plasmid also carries an antibiotic resistance gene, such as but not limited to kanamycin.

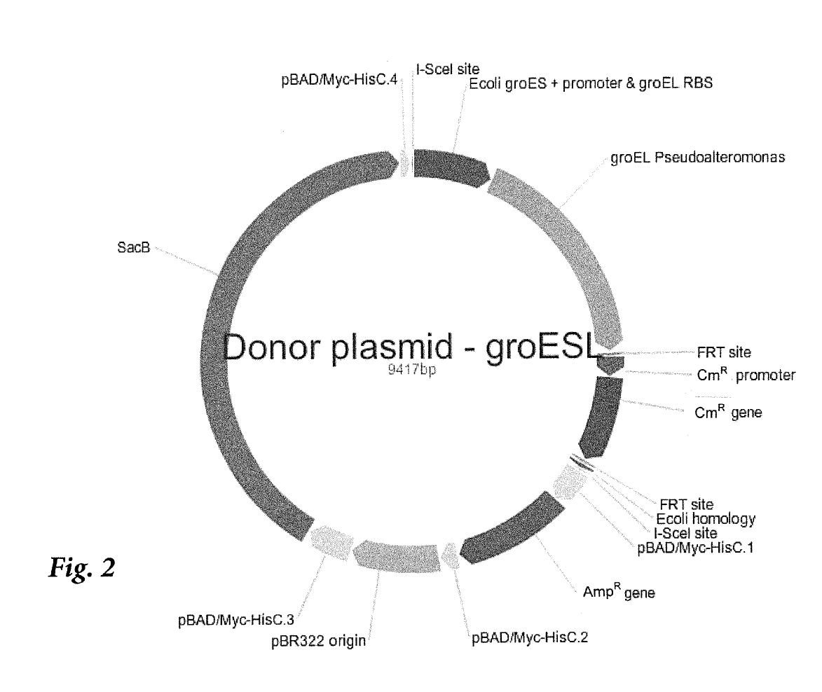

The donor plasmid. Double-stranded linear DNA—as produced in a polymerase chain reaction (PCR)—is degraded by exonucleases when transformed directly into competent E. coli cells. Therefore, a different means of introducing the gene cluster into the E. coli host cells was developed as part of the recombineering method of the present disclosure and involves the use of a donor plasmid. The donor plasmid shall next be described with reference to FIG. 2.

The Gibson Assembled gene cluster, or a knockout gene sequence, pursuant to the method of the present disclosure is cloned into pBAD, the donor plasmid, which is transformed into competent E. coli host cells. The gene cluster is designed such that an S. cerevisiae I-SceI recognition site flanks each end of the Gibson Assembled gene cluster. Either a gene cluster or a knockout gene sequence is delivered into the E. coli cells via the donor plasmid. This occurs when the cluster (or knockout gene sequence) is excised out of the donor plasmid by the I-SceI endonuclease as it cleaves the flanking recognition sites.

The donor plasmid also carries, as part of the gene cluster/knockout gene sequence, an antibiotic resistance gene, such as but not limited to chloramphenicol, which is flanked by FRT (FLP recognition target) sites. The conferred antibiotic resistance allows for selection, for if recombination is successful and the gene cluster/knockout gene sequence is inserted into the E. coli genome, the cells will be resistant to that antibiotic. Once recombination of the gene cluster/knockout gene sequence into the genome has been verified, the antibiotic resistance gene can be excised by the FLP recombinase protein that cleaves and ligates at the FRT sites. The FLP recombinase protein is coded for by the flp gene cloned into the helper plasmid.

The identification of recombinants (cells containing gene clusters in the host cell genome) is more efficient and reproducible when using sacB counter-selection. The sacB gene makes E. coli sensitive to sucrose; thus, plates containing sucrose can be used to select against cells containing this functioning gene. Counter-selection is used post-recombination and after screening for antibiotic resistance conferred by the antibiotic resistance gene that is incorporated via the gene cluster/knockout gene sequence into the genome. If the plasmid carrying the sacB gene was not successfully cleaved by the I-SceI endonuclease, then cells will not grow on plates containing sucrose. The sacB gene will be included in the donor plasmid and the helper plasmid.

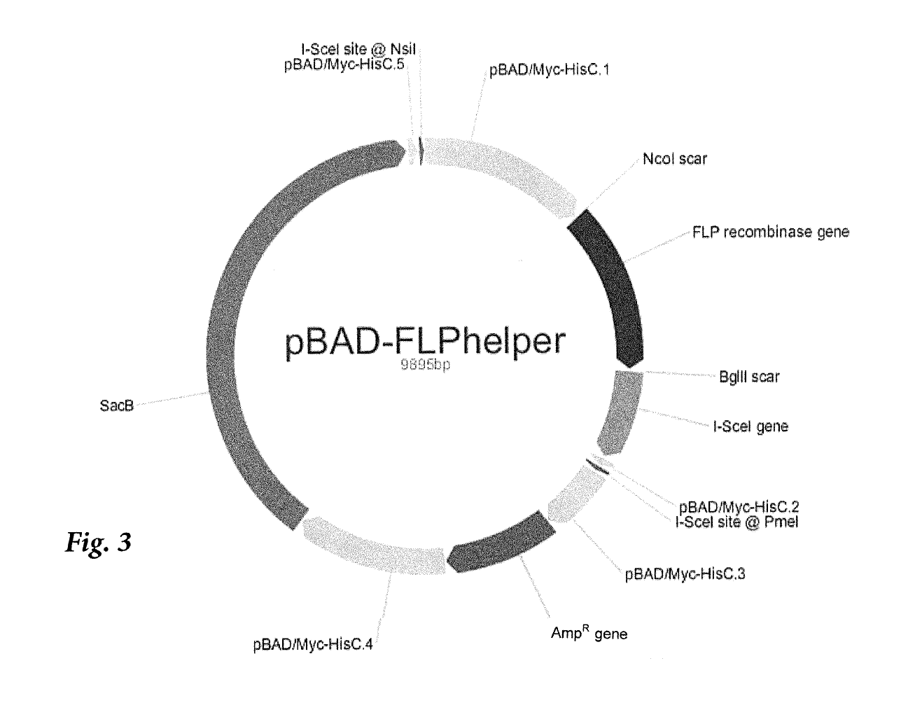

The helper plasmid. After selection for the antibiotic resistance gene within the gene cluster/knockout gene sequence, the resistance gene will be eliminated by using a helper plasmid that expresses the FLP recombinase protein. The helper plasmid shall next be described with reference to FIG. 3. The FLP recombinase acts on FRT (FLP recognition target) sites flanking the resistance gene within the gene cluster or knockout gene sequence. The helper plasmid also carries an antibiotic resistance gene, such as but not limited to ampicillin. And, like the donor and λ-red plasmids, the helper plasmid contains the I-SceI gene, which codes for the I-SceI protein. There are also two I-SceI recognition sites located in the helper plasmid, which allow for self-cleavage of this plasmid. The purpose of cleaving the helper plasmid is to prevent over-expression of the FLP recombinase protein and to eliminate unnecessary plasmids from the host cells.

Cloning PUFA genes. The Shewanella pneumataphori pfaA, pfaB, pfaC, and pfaD, and particularly Shewanella pneumataphori SCRT-2738 pfaA, pfaB, pfaC, and pfaD genes of the preferred embodiment can be cloned in a pBad plasmid using restriction sites XhoI and PmeI. The Moritella marina pfaE, and particularly Moritella marina MP-1 pfaE genes of the preferred embodiment can be cloned into a pBAD plasmid using restriction sites XhoI and SnaBI.

The Polyketide Synthesis (PKS) pathway. In the cellular production of PUFAs, the polyketide synthesis pathway involves seven protein domains, coded by five genes in Shewanella pneumatophori SCRC-2738. Known to be very versatile in its products, the polyketide synthesis pathway is not fully understood and functions differently based on the organism and the end-product molecule.

PKS genes of the preferred embodiment. Four polyunsaturated fatty acid (PUFA) producing genes native to the cold water Pacific bacterium Shewanella pneumatophori SCRC-2738 and one from Moritella marina MP-1 are cloned into an E. coli strain modified for increased EPA output. Four of these, pfaA-D, which are to be expressed in the heterologous production of EPA, are arranged consecutively in the EPA-producing gene cluster of Shewanella pneumatophori, while pfaE is cloned from Moritella marina MP-1. Although the exact protein assembly of pfaA-D is not well understood, the protein domains have been identified as being homologous to other known PKS proteins, as well as a few fatty acid synthesis (FAS) proteins. Especially of note in the multifunctional PfaA protein are the six domain repeats of ACP, which is thought to be correlated with high PUFA production.

The M. marina pfaE gene is homologous to the pfaE gene of S. Pneumataphori. It has been shown that the Moritella marina pfaE gene product works with the S. pneumatophori pfaA-D gene cluster product (enzymes) to produce EPA in E. coli cells. The pfaE gene from Moritella marina codes for 4′-phosphopantetheinyl transferase, or PPTase, which catalyzes the transfer of a phosphopantetheinyl group from intracellular CoASH (reduced coenzyme A) to the active site of an acyl carrier protein (ACP); this is the first step in the cellular production of fatty acids, including EPA. The extent of this post-translational modification of ACP within cells controls the rate and yield of produced fatty acids.

For EPA synthesis, 4′-phosphopantetheinyl transferase (PPTase) transfers a phosphopantetheinyl group onto the acyl carrier protein (ACP), transforming the ACP from its inactive apo-ACP form to the active holo-ACP form. A 3-ketoacyl-ACP synthase (KS) catalyzes a condensation reaction in coordination with the ACP and the attached carbon elongation unit (usually an acetyl or malonyl group) to synthesize a carbon-carbon bond and lengthen the growing polyketide. Then the 3-ketoacyl-ACP reductase (KR) reduces the beta ketone to a hydroxyl group, and the 3-hydroxydecanoyl-ACP dehydratase (DH) dehydrates the hydroxyl, leaving a carbon-carbon double bond. It is thought that this hydratase also has an isomerase function (DH/I) to convert the trans-double bond to a cis-conformation, which is biologically favored. Finally, the enoyl-ACP reductase further reduces the double bond to a fully saturated single bond, where applicable, before adding the next iterative two-carbon unit until the chain reaches twenty carbons.

Codon bias of heterologous genes. Whenever introducing genetic material into a heterologous host with the purpose of expressing those genes and increasing the yield of the protein product, codon use frequency should be considered. Codon use may be a strong predictor of gene expression. Charged transfer RNAs, or tRNAs ‘loaded’ with an amino acid and ready for translation, may be a limiting reactant for heterologous expression, especially during periods of amino acid starvation or high translational activity. Some tRNAs are less sensitive to starvation than others, and these will be charged at a much higher frequency than other tRNAs that code for the same amino acid. Designing the codon usage of the heterologous genes accordingly will make expression more robust.

A useful tool for the development of sequence models is Gene Designer 2.0 available commercially from DNA 2.0, Menlo Park, Calif. The DNA fragments designed by this software tool can then be ordered using Gene Designer files.

Preferred growth conditions for the recombinant host of the present disclosure. For the purpose of producing EPA with maximal yield, optimal growing conditions must be utilized. The following conditions are proposed to achieve the goal of EPA production with high yields.

Growing at low temperature. It has been noted that heterologous expression of PKS proteins in E. coli can result in an accumulation of inclusion bodies; however, control of temperature, pressure, and medium composition has yielded substantial levels of correctly folded PKS proteins. Thus, for the purpose of producing EPA with maximal yield, optimal growing conditions must be utilized.

Expression of heterologous genes in E. coli often results in the production of proteins that are insoluble, inactive, or rapidly degraded. Aggregates of these proteins, known as inclusion bodies, are thought to result from the proteins' inability to fold into their native state. A technique to limit inclusion body formation is to grow cells at reduced temperatures. Because the optimal temperature for the psychrophilic S. pneumatophori EPA-producing enzymes is 15° C., growing the cells at a low temperature to minimize inclusion body formation is cooperatively beneficial. Unfortunately, because E. coli is a mesophilic organism, cultivation at reduced temperatures results in a dramatic reduction in the growth rate and, consequently, the reduced rate of the synthesis of heterologous proteins. Specifically, E. coli growth is impaired at temperatures below 21° C. and stops at 7.5° C. Reasons for reduced growth rates were revealed in experiments conducted by Strocchi et al., Results identified twenty-two housekeeping proteins that are involved in systems failure of E. coli when grown at low temperatures. Specifically, experiments suggested that the mechanism of cold-induced failure in E. coli is the result of the inactivation of GroEL/GroES chaperonins at the low temperatures. The inactivated GroEL/GroES chaperonins fail to refold the denatured housekeeping proteins, thus leading to systems failure in E. coli when grown at low temperatures. Experiments have determined that the E. coli GroEL chaperonin consists of a homotetradecameric double-ring cylinder composed of ˜57 kDa subunits and that its co-chaperonin, GroES, consists of a homoheptameric dome-shaped ring composed of ˜10 kDa subunits. Also shown was that the GroEL subunit consists of an apical, an intermediate, and an equatorial domain. The apical domains form the entrance to the GroEL cavity and include the residues involved in binding to GroES and unfolded proteins. The small intermediate domain has potential hinge regions at its connection to the equatorial and apical domains. The equatorial domain, which includes both the N- and C-termini of GroEL, contains an ATP-binding site and also most of the residues that make inter-subunit contacts. Furthermore, it was determined that the C-terminal segment of the GroEL equatorial domain plays an important role in inter-subunit interactions in GroEL assembly and that the C-terminal segment is not highly conserved. Thus, when the DNA sequence that codes for the C-terminal segment of GroEL from the cold-adapted psychrophilic bacterium Pseudoalteromonas sp. PS1M3 was introduced into E. coli in the form of a chimeric plasmid construct, the E. coli cells became cold-adapted. In other words, the expression of the cold-adapted GroEL in E. coli resulted in a shortened doubling time of the cells when grown at low temperatures. Based on these results, it is proposed to clone the Pseudoalteromonas sp. PS1M3 groEL chaperonin gene into the E. coli genome to facilitate growth of the EPA-producing cells at 15° C. and subsequently minimize the formation of heterologous protein inclusion bodies. The groESL promoter region, groES gene, and the approximately 49 by segment between groES and groEL will be cloned from E. coli NEB-10β cells (obtained from New England Biolabs in a preferred embodiment); this sequence will be followed by the Pseudoalteromonas sp.PS1M3 groEL gene sequence. Gene expression will be regulated by both E. coli sigma-70 and sigma-32 promoter sequences for constitutive expression and production of the cold-adapted GroEL/GroES proteins.

Media composition. Culturing the recombinant E. coli cells in corn steep liquor gave good EPA productivity and would reduce overall fermentation costs. It has been shown that corn steep liquor has significant pantothenic acid (vitamin B₅) content, which would provide the required pantothenic acid supply for the host cells.

Thus, the present invention is well adapted to carry out the objects and attain the ends and advantages mentioned above as well as those inherent therein. While presently preferred embodiments have been described for purposes of this disclosure, numerous changes and modifications will be apparent to those skilled in the art. Such changes and modifications are encompassed within the spirit of this invention as defined by the appended claims.

What is claimed is:

1. A method of producing eicosapentaenoic acid (EPA) in a recombinant bacterial host, the method comprising:
a. selecting a bacterial host including at least one biosynthetic pathway, at least one degradation pathway, and at least one metabolic pathway;
i. deleting said at least one biosynthetic pathway;
ii. deleting said at least one degradation pathway;
iii. deleting said at least one metabolic pathway;
b. inserting genes into said bacterial host, said genes selected from the group consisting of Escherichia coli panK, Bacillus subtilis sfp, Moritella marina putative thioesterase I, Pseudoalteromonas sp. GroEL, and E. coli GroES to produce a recombinant host;
c. expressing in said bacterial host genes cloned into a first pBAD; said genes selected from the group consisting of Shewanella pneumataphori pfaA, pfaB, pfaC, and pfaD;
d. growing said recombinant host to optimize EPA production.

2. The method of claim 1 wherein said bacterial host is Escherichia coli.

3. The method of claim 2 wherein said Escherichia coli is of the strain NEB-10β.

4. The method of claim 2 wherein said recombinant host is grown at low temperatures.

5. The method of claim 4 wherein said low temperatures are less than about 16° C.

6. The method of claim 4 wherein said low temperatures are between about 13° C. and 16° C.

7. The method of claim 4 wherein said low temperatures are between about 14° C. and 15° C.

8. The method of claim 4 wherein said recombinant host is cultured in corn steep liquor.

9. The method of claim 1 wherein said at least one biosynthetic pathway is at least one fatty acid biosynthesis gene.

10. The method of claim 1 wherein said at least one degradation pathway is at least one fatty acid degradation gene.

11. The method of claim 1 wherein said at least one metabolic pathway is at least one E. coli pgi gene and at least one E. coli pta gene.

12. The method of claim 1 further including expressing in said bacterial host Moritella marina pfaE genes cloned into a second pBAD.

13. The method of claim 12 further including over-expressing the panK gene.

14. The method of claim 9 wherein said at least one fatty acid biosynthesis gene is fabB.

15. The method of claim 10 wherein said at least one fatty acid degradation gene includes fadD and fadE.

16. A method of producing eicosapentaenoic acid (EPA) in a recombinant bacterial host, the method comprising:
a. selecting an E. coli bacterial host including at least one fatty acid biosynthesis gene, at least one fatty acid degradation gene, and at least one phosphate acetyl transferase gene;
i. deleting said at least one fatty acid biosynthesis gene;
ii. deleting said at least one fatty acid degradation gene;
iii. deleting said at least one phosphate acetyl transferase gene;
b. expressing genes in said bacterial host genes selected from the group consisting of Escherichia coli panK, Bacillus subtilis sfp, Moritella marina putative thioesterase I, Pseudoalteromonas GroEL, and E. coli GroES to produce a recombinant host;
c. expressing in said bacterial host genes cloned into a first pBAD; said genes selected from the group consisting of Shewanella pneumataphori SCRT-2738 pfaA, pfaB, pfaC, and pfaD;
d. expressing in said bacterial host a Moritella marina MP-1 pfaE genes cloned into a second pBad;
e. growing said recombinant host at low temperatures to optimize EPA production.

17. The method of claim 16 wherein said low temperatures are approximately 14° C. and 15° C.

18. The method of claim 16 wherein said at least one fatty acid biosynthesis gene is E. coli fabB.

19. The method of claim 16 wherein said at least one fatty acid degradation gene includes E. coli fadD and E. coli fadE.

20. The method of claim 16 wherein said at least one phosphate acetyl transferase gene is E. coli pta and E. coli pgi and further including the over-expression of E. coli panK.
